(19) United States
(12) Patent Application Publication
(10) Pub. No.: US 2017/0304297 A1
(43) Pub. Date: Oct. 26, 2017

(54) IMMEDIATE RELEASE ABUSE DETERRENT TABLET

(71) Applicant: Allergan Sales, LLC, Irvine, CA (US)

(72) Inventors: Rahul Sareen, Weston, FL (US); Shahin Fesharaki, Weston, FL (US); Parag Shah, Weston, FL (US)

(73) Assignee: Allergan Sales, LLC, Irvine, CA (US)

(21) Appl. No.: 15/640,823

(22) Filed: Jul. 3, 2017

Related U.S. Application Data
(63) Continuation of application No. 14/357,814, filed on May 13, 2014.

(51) Int. Cl. A61K 31/485; A61K 9/20; A61K 9/46

(52) U.S. Cl. CPC A61K 31/485; A61K 9/2031; A61K 9/2013; A61K 9/2054; A61K 9/0007; A61K 9/2018; A61K 9/2059

(57) ABSTRACT

The invention relates to an abuse deterrent immediate release tablet.

### FIELD OF THE INVENTION

The present invention relates to the field of solid oral dosage forms and in particular solid immediate release tablets that contain a drug which is subject to abuse. The solid oral dosage forms of the present invention contain an effervescent agent and are designed to release substantially all of the drug in about 15 to about 60 minutes.

### BACKGROUND OF THE INVENTION

Although pharmaceutical dosage forms are designed to be safe and effective in the treatment of various afflictions, they are sometimes misused and subject to illicit use and/or abuse by individuals. Some of the more commonly misused pharmaceutical dosage forms contain opioids, sedatives, stimulants and hypnotics. One of the more common illicit practices is to obtain solid dosage forms such as tablets or capsules and manipulate the solid dosage form, typically by crushing the solid dosage form, and to extract the drug to allow the drug to be administered by inhalation or injection.

Due to the increased illicit use of pharmaceutical products there has been an effort to design solid oral dosage forms that will hopefully deter or prevent their misuse and abuse. For example United States Published Patent Application Nos. 2005/0031546 and 2009/0081290 describe crush resistant opioid dosage forms that contain polyethylene oxide. United States Published Patent Application No. 2010/0015223 describes hard opioid matrix tablets that release the opioid drug over extended periods of time and that are difficult to crush. Another method described in the art to deter illicit use of pharmaceutical products is to include aversive agents such as irritating agents for the nasal and/or pharyngeal tracts, antagonist agents for the drug that is being abused, bittering agents, visual modifying agents, emetic agents and viscosity increasing agents. Examples of abuse deterrent dosage forms employing irritating agents and/or emetic agents are described in U.S. Pat. No. 7,510,726. Examples of abuse deterrent dosage forms employing antagonist agents are described in U.S. Pat. Nos. 7,682,633 and 7,658,939. Examples of abuse deterrent dosage forms employing viscosity increasing agents are described in U.S. Pat. No. 7,776,314 and European Patent Application No. 0 661 045. Examples of abuse deterrent dosage forms employing visual modifying agents are described U.S. Pat. No. 6,514,531, and examples of abuse deterrent dosage forms employing bittering agents are provided in U.S. Pat. No. 7,141,250.

Although the art contains many examples of abuse deterrent dosage forms there is a constant need to improve the deterrents to abuse and to prepare a safe and effective solid dosage form in a simple, easy and cost effective manner.

### SUMMARY OF THE INVENTION

It is an object of the present invention to provide an improved abuse deterrent solid oral dosage form that is safe, effective and easy to manufacture.

It is a further object of the present invention to provide an immediate release tablet which is subject to less abuse, preferably less intranasal abuse.

It is a further object of the present invention to provide a method for preparing immediate release tablets, preferably immediate release opioid tablets, which are subject to less abuse.

These and other objectives are achieved by a solid pharmaceutical tablet comprising:

a) a therapeutically effective amount of a drug that is subject to abuse;

b) about 1 to about 20 weight percent of a gelling agent; and

c) about 1 to about 20 weight percent of an effervescent agent wherein the tablet releases substantially all of the drug in about 15 to about 60 minutes when placed into 500 ml of an aqueous media. It is believed that the gelling agent will make extraction of the drug substance from the crushed tablet difficult and the effervescent agent will act as a nasal irritant that will deter inhalation.

Embodiments of tablets prepared in accordance with the present invention may further comprise conventional pharmaceutical processing excipients selected from the group consisting of fillers, binders, lubricants, glidants, disintegrants, coloring agents and mixtures thereof.

Embodiments of the present invention may also further comprise additional aversive agents such as a second or additional irritating agent for the nasal and/or pharyngeal tracts, antagonist agents for the drug that is being abused, bittering agents, visual modifying agents, emetic agents and combinations of the forgoing.

One embodiment of the present invention will comprise a second nasal irritant that is different from the effervescent agent. In this embodiment the second nasal irritant may comprise about 0.5 to about 10 weight percent of tablet.

A further embodiment of the present invention comprises a method for preparing an immediate release abuse deterrent tablet that comprises the steps of:

a) dry mixing:

i) a therapeutically effective amount of a drug that is subject to abuse;

ii) about 1 to about 20 weight percent of a gelling agent;

iii) about 1 to about 20 weight percent of an effervescent agent;

iv) optionally about 1 to about 10 weight percent of a second nasal irritant other than the effervescent agent; and

v) optionally conventional pharmaceutical processing excipients selected from the group consisting of fillers, binders, lubricants, glidants, disintegrants, coloring agents and mixtures thereof; and

b) compressing the dry mixture into tablets that release substantially all of the drug in about 15 to about 60 minutes when placed into 500 ml of an aqueous media.

### BRIEF DESCRIPTION OF THE DRAWINGS

FIG. 1 is the dissolution profile of the 5 mg tablet of Example 3.

FIG. 2 is the dissolution profile of the 7.5 mg tablet of Example 3.

FIG. 3 is the dissolution profile of the 5 mg tablet of Example 4.

FIG. 4 is the dissolution profile of the 7.5 mg tablet of Example 4.

FIG. 5 is the dissolution profile of the 7.5 mg tablet of Example 5.

FIG. 6 is the dissolution profile of the 5 mg tablet of Example 6.

FIG. 7 is the dissolution profile of the 7.5 mg tablet of Example 6.

### DETAILED DESCRIPTION OF THE INVENTION

The present invention is an immediate release tablet with abuse deterrent characteristics and a method for making the immediate release tablet. The tablet comprises:

a) a therapeutically effective amount of a drug that is subject to abuse, preferably comprising about 0.5 weight percent to about 75 weight percent of the total tablet weight, preferably about 1 weight percent to about 50 weight percent of the total tablet weight;

b) a gelling agent comprising about 1 weight percent to about 20 weight percent of the total tablet weight, preferably about 3 weight percent to about 15 weight percent of the total tablet weight;

c) an effervescent agent comprising about 1 weight percent to about 20 weight percent of the total tablet weight, preferably about 2 weight percent to about 10 weight percent of the total tablet weight; and

d) optionally a second nasal irritant other than the effervescent agent comprising about 0.5 weight percent to about 10 weight percent of the total tablet weight, preferably about 1 weight percent to about 5 weight percent of the total tablet weight.

The tablets of the present invention should release substantially all of the drug in about 15 to about 60 minutes when placed into 500 ml of an aqueous media. The tablets should release the drug in a pH independent manner meaning the drug should be released from the tablet at substantially the same rate regardless of the pH of the aqueous media. The release can be measured according to the procedures described in section <711> DISSOLUTION of the United States Pharmacopeia.

One embodiment of the present invention will release the drug with the following profile:

| Time | Preferred % Release | Most Preferred % Release |
|---|---|---|
| 30 min | 20-75% | 30-70% |
| 45 min | 60-100% | 75-100% |

Another embodiment of the present invention will release the drug with the following profile:

| Time | Preferred % Release | Most Preferred % Release |
|---|---|---|
| 30 min | 40-90% | 50-80% |
| 45 min | 70-100% | 80-100% |

Examples of drugs that are subject to abuse and that may be used in the present invention include opioids, tranquilizers, sedatives and stimulants. Examples of such drugs can be found on pages 1487-1555 of Remington, The Science and Practice of Pharmacy, 21^{st} ed. (2005). Specific examples of the drugs that may be used in the present invention include alfentanil, alimemazine, alprazolam, amphetamine, buprenorphine, butorphanol, clonazepam, codeine, cyclobenazprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, zolpidem or pharmaceutically acceptable salts thereof. Specific embodiments of the present invention comprise opioids selected from the group consisting of buprenorphine, codeine, dihydrocodeine, dihydromorphine, hydrocodone, hydromorphone, morphine, oxycodone, oxymorphone or pharmaceutically acceptable salts therefore.

The therapeutic amounts of the drugs used in the present invention are known in the art and can be found in the literature such as Goodman & Gillman's, The Pharmacological Basis of Therapeutics, 9^{th} ed. pages 219-222, 361-396, 521-535. For example, typical therapeutic amounts of hydromorphone range from about 4 mg to about 64 mg of the hydrochloride salt, typical therapeutic amounts of morphine range from about 5 mg to about 800 mg and typical therapeutic amount of oxycodone range from about 5 mg to abut 400 mg for the hydrochloride salt.

The gelling agent that may be employed in the tablets of the present invention is a material that exhibits the ability to retain a significant fraction of imbibed fluid in the molecular structure. The gelling agents are materials that can swell or expand when in contact with an aqueous media such as gastric or intestinal fluid to a very high degree. The swelling or expansion of the gelling agent usually exhibits a 2 to 50 fold volume increase from the dry state. Examples of gelling agents that may be used in the present invention include swellable polymers, also known as osmopolymers or hydrogels. The swellable polymer can be non-cross-linked or lightly cross-linked. The cross-links can be covalent or ionic bonds with the polymer possessing the ability to swell in the presence of fluid, and when cross-linked it will not be dissolved in the fluid. The polymer can be of plant, animal or synthetic origin. Polymeric materials useful for the present purpose include polyhydroalkylcellulose having a molecular weight greater than 50,000 poly(hydroxyalkylmethacrylate) having a molecular weight of from 5,000 to 5,000,000; poly(vinylpyrrolidone) having a molecular weight of from 100,000 to 3,000,000; anionic and cationic hydrogels; poly(electrolyte) complexes; poly(vinylalcohol) having a low acetate residual; a swellable mixture of agar and carboxymethyl cellulose; a swellable composition comprising methyl cellulose mixed with a sparingly cross-linked agar; a polyether having a molecular weight of from 10,000 to 6,000,000; water swellable copolymer produced by a dispersion of finely divided copolymer of maleic anhydride with styrene, ethylene, propylene, or isobutylene; water swellable polymer of N-vinyl lactams; and the like.

Other gelling agents useful in the present invention include pectin having a molecular weight ranging from 30,000 to 300,000; polysaccharides such as agar, acacia, karaya, tragacanth, algins and guar; Carbopol®, an acrylic acid polymer, a carboxyvinyl polymer, sometimes referred to as carboxypolymethylene, a polymer of acrylic acid cross-linked with a polyallyl ether of sucrose, as described in U.S. Pat. Nos. 2,798,053 and 2,909,462 and available as Carbopol® 934, 940 and 941, and its salt derivatives; polyacrylamides; water-swellable indene maleic anhydride polymers; Good-rite® polyacrylic acid having a molecular weight of 80,000 to 200,000; Polyox® polyethylene oxide polymers having a molecular weight of 100,000 to 7,000,000; starch graft copolymers; Aqua-Keep® acrylate polymers with water absorbability of about 400 times its original weight; diesters of polyglucan; a mixture of cross-linked polyvinyl alcohol and poly(N-vinyl-2-pyrrolidone); poly(ethylene glycol) having a molecular weight of 4,000 to 100,000. Representative polymers possessing gelling properties are described in U.S. Pat. Nos. 6,419,954, 4,915,949, 4,327,725, 4,207,893 and in Handbook of Common Polymers, by Scott and Roff, published by Cleveland Rubber Company, Cleveland, Ohio.

One embodiment of the present invention employs polyethylene oxide as the gelling agent. The polyethylene oxide should have an approximate molecular weight of about 100,000 to 7,000,000, preferably between about 500,000 and about 5,000,000 and most preferably about 900,000 to about 5,000,000.

An alternative embodiment of the present invention employs a combination of two or more gelling agents preferably selected from the group consisting of polyethylene oxide, hydroxypropyl cellulose with a molecular weight of about 50,000 to about 125,000, hydroxypropyl methylcellulose with a 2% (w/v) aqueous viscosity at 20° C. between about 50 mPa·s and about 100,000 mPa·s, and polyvinylpyrrolidone with a molecular weight between 400,000 to about 3,000,000. One embodiment of the present invention employs a mixture of at least two different types of polyethylene oxides wherein the first polyethylene oxides has an approximate molecular weight between 500,000 and 1,000,000 and the second polyethylene oxide has an approximate molecular weight between 2,000,000 and 5,000,000. The ratio of the first, or lower, molecular weight polyethylene oxide to the second, or higher, molecular weight polyethylene oxide is about 1:5 to about 5:1 preferably about 1:4 to about 4:1.

The effervescent agent employed in the present invention is a material or combination of materials that evolve, gas by means of a chemical reaction that takes place upon the exposure of the tablet to an aqueous media such as saliva, gastric fluid or intestinal fluid. The gas generating reaction is typically the result of a reaction between an acid source and an alkaline source. The acid source should be an acid that is safe for human consumption and generally includes organic acids such as citric acid, tartaric acid, malic acid, maleic acid, lactic acid, glycolic acid, ascorbic acid, fumaric acid, adipic acid, succinic acid and combinations thereof. Acid salts such as sodium dihydrogen phosphate, disodium hydrogen phosphate, sodium acid sulfite and metal salts of citric acid, tartaric acid, malic acid, maleic acid, lactic acid, glycolic acid, ascorbic acid, fumaric acid, adipic acid and succinic acid, such as sodium citrate or sodium tartrate, may also be employed as the acid source of the effervescent agent. The alkaline source should release oxygen or carbon dioxide gas when reacting with the acid source. One embodiment of the alkaline source is a material that will release carbon dioxide when reacted with the acid source such as carbonates and bicarbonates. Examples of the carbonates and bicarbonate that may be used in the present invention include sodium bicarbonate, sodium carbonate, potassium bicarbonate, potassium carbonate, magnesium carbonate, sodium sequicarbonate, sodium glycine carbonate, L-lysine carbonate, arginine carbonate, calcium carbonate, calcium bicarbonate and mixtures of the foregoing. Another embodiment of the alkaline source is a material that will release oxygen. Examples of compounds that will release oxygen are anhydrous sodium perborate, effervescent perborate, sodium perborate monohydrate, sodium precarboante and mixtures thereof.

Embodiments of tablets prepared in accordance with the present invention may further comprise conventional pharmaceutical excipients selected from the group consisting of fillers, binders, lubricants, glidants, disintegrants, coloring agents and mixtures thereof. A more detailed description of the pharmaceutical excipients that may also be included in the tablets of the present invention can be found in The Handbook of Pharmaceutical Excipients, 5^{th} ed. (2006).

Examples of fillers that may be employed in the present invention include, but are not limited to lactose, starch, dextrose, sucrose, fructose, maltose, mannitol, sorbitol, kaolin, microcrystalline cellulose, powdered cellulose, dextrates, calcium sulfate, calcium phosphate, dicalcium phosphate, lactitol or any combination of the foregoing. The amount of filler that may be employed in the tablets of the present invention is generally about 5 weight percent to about 75 weight percent based upon the total weight of the tablet and preferably about 10 weight percent to about 50 weight percent based upon the total weight of the tablet.

Examples of binders that may be employed in the present invention include, but are not limited to acacia, alginic acid, sodium carboxymethylcellulose sodium, microcrystalline cellulose, dextrin, ethylcellulose, gelatin, starch, polyvinyl alcohol, polyethylene oxide, polyvinylpyrrolidone, methylcellulose, hydroxypropyl cellulose, hydroxymethyl cellulose and any combination of the foregoing. The binders may be water soluble materials. The binders also may exhibit a low molecular weight and/or low viscosity when measured in a 2% aqueous solution. The low molecular weight binders typically are polymers with a molecular weight of less than 50,000, preferably less than 30,000, and preferably less than 10,000. The low viscosity binders typically have a viscosity of about 50 mPa·s or lower, preferably about 25 mPa·s or lower and most preferably 15 mPa·s or lower. The amount of binder that may be employed in the tablets of the present invention is generally about 1 weight percent to about 25 weight percent based upon the total weight of the tablet and preferably about 3 weight percent to about 15 weight percent based upon the total weight of the tablet.

Examples of lubricants that may be used in the present invention include, but are not limited to talc, glyceryl monostearates, calcium stearate, magnesium stearate, stearic acid, glyceryl behenate, polyethylene glycol, poloxamer and combinations of the foregoing. The amount of lubricant that may be employed in the tablets of the present invention is generally about 0.05 weight percent to about 15 weight percent based upon the total weight of the tablet, preferably about 0.1 weight percent to about 10 weight percent based upon the total weight of the tablet and most preferably about 0.5 weight percent to about 5 weight percent based upon the total weight of the tablet.

Examples of glidants that may be used in the present invention include, but are not limited to colloidal silicon dioxide (Cab-O-Sil) and Quso (also known as Phila Quartz). The amount of glidant that may be employed in the tablets of the present invention is generally about 0.1 weight percent to about 7.5 weight percent based upon the total weight of the tablet and preferably about 0.5 weight percent to about 5 weight percent based upon the total weight of the tablet.

Examples of disintegrants that may be used in the present invention include, but are not limited to corn starch, croscarmellose sodium, crospovidone (polyplasdone XL-10), sodium starch glycolate (EXPLOTAB® or PRIMOJEL®) or any combination of the foregoing. The amount of disintegrant that may be employed in the tablets of the present invention is generally about 0.5 weight percent to about 10 weight percent based upon the total weight of the tablet and preferably about 1 weight percent to about 5 weight percent based upon the total weight of the tablet.

Coloring agents that may be employed in the present invention include FD&C-type dyes and lakes, fruit and vegetable extracts, titantium dioxide and mixtures thereof. The coloring agent can be incorporated into the tablet matrix by blending the coloring agent with the drug, gelling agent and effervescent agent. Alternatively, the coloring agent may be applied to the outer surface of the tablet as part of an aesthetic coating or seal coating.

The tablet of the present invention can be made by any means commonly used in the pharmaceutical arts. Embodiments of the present invention can be prepared by granulating the tablet ingredients and compressing the granules into a tablet. The granules may be prepared by wet granulation or dry granulation techniques. A dry granulation technique may include a slugging step and/or roller compaction. If a wet granulation step is employed in the method for preparing the tablets of the present invention, the wet granules should be prepared and dried before the effervescent agent is added to the granules. For example, in one embodiment, the drug and a filler and/or a binder may be wet granulated using a fluidized bed granulator. The granules can be collected, dried and sized. The dried granules will be mixed with the gelling agent, the effervescent agent and a lubricant and/or glidant and the resulting mixture compressed into a tablet. In an alternate embodiment all the components of the tablet are dry blended and the dry blend is compressed into a tablet. The hardness of the tablet of the present invention should be from about 5 to about 20 kp, preferably about 10 to about 15 kp.

The present invention may also include aversive agents in addition to the effervescent agent. The aversive agents may include a second irritating agent for the nasal and/or pharyngeal tracts, antagonist agents for the drug that is being abused, bittering agents, visual modifying agents, emetic agents and combinations of the forgoing.

The nasal and/or pharyngeal irritants include compounds generally considered pharmaceutically inert, yet can induce irritation, such compounds include, but are not limited to surfactants such as sodium lauryl sulfate, poloxamer, sorbitan monoesters and glycerol monooleates.

Antagonist agents include compounds that block or negate the effect the drug. Examples of antagonist agents for opioids include compounds such as naltrexone, naloxone, nalmefene, cyclazacine, levallorphan. Specific examples of antagonist agents and methods for preparing the antagonist agent for incorporation into the tablets of the present invention are provided in U.S. Pat. Nos. 7,682,633 and 7,658,939, which are incorporated herein by reference.

The visual modifying agents and emetic agents may also be included in the present invention. Examples of the visual modifying agents are provided in U.S. Pat. No. 6,514,531 and Examples of emetic agents are provided in U.S. Pat. No. 7,510,726, which are incorporated herein by reference.

The following examples illustrate the present invention and are not intended to limit the scope of the present invention.

### Example 1

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.02%) | 7.50 (1.53%) |
| Polyethylene oxide | 25.00 (5.10%) | 25.00 (5.10%) |
| (POLYOX WSR 1105*) | | |
| Microcrystalline cellulose | 274.50 (56.02%) | 272.00 (55.51%) |
| (AVICEL PH 102) | | |
| Sodium lauryl sulfate, NF | 7.00 (1.43%) | 7.00 (1.43%) |
| Corn Starch, NF | 100.00 (20.41%) | 100.00 (20.41%) |
| Sodium Bicarbonate, USP | 50.00 (10.20%) | 50.00 (10.20%) |
| Citric Acid, USP | 25.00 (5.10%) | 25.00 (5.10%) |
| Colloidal Silicon Dioxide, NF | 2.50 (0.51%) | 2.50 (0.51%) |
| (Cab-O-Sil M5P) | | |
| Magnesium Stearate, NF | 1.00 (0.20%) | 1.00 (0.20%) |
| Total | 490.00 | 490.00 |

*approximate molecular weight of 900,000

The tablets were prepared by screening the oxycodone hydrochloride and polyethylene oxide through a 30 mesh screen, loading the screened material into a V-blender and blending the material for approximately 5 minutes. Approximately one third of the microcrystalline cellulose was screened through a 30 mesh screen and loaded into the V-blender and blended for approximately 10 additional minutes. The remaining microcrystalline cellulose, sodium lauryl sulfate, corn starch, sodium bicarbonate, citric acid and colloidal silicon dioxide was screened through a 30 mesh screen and loaded into the V-blender and blended for approximately 10 additional minutes. The magnesium stearate was screened through a 30 mesh screen and loaded into the V-blender and blended for approximately 5 additional minutes. The final blend is compressed into tablets with a target hardness of about 5 to 15 kp, preferably with a target hardness of about 12 kp.

### Example 2

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.02%) | 7.50 (1.53%) |
| Polyethylene oxide (POLYOX | 25.00 (5.10%) | 25.00 (5.10%) |
| WSR Coagulant fine powder*) | | |
| Microcrystalline cellulose | 274.50 (56.02%) | 272.00 (55.51%) |
| (AVICEL PH 102) | | |
| Sodium lauryl sulfate, NF | 7.00 (1.43%) | 7.00 (1.43%) |
| Corn Starch, NF | 100.00 (20.41%) | 100.00 (20.41%) |
| Sodium Bicarbonate, USP | 50.00 (10.20%) | 50.00 (10.20%) |
| Citric Acid, USP | 25.00 (5.10%) | 25.00 (5.10%) |
| Colloidal Silicon Dioxide, NF | 2.50 (0.51%) | 2.50 (0.51%) |
| (Cab-O-Sil M5P) | | |
| Magnesium Stearate, NF | 1.00 (0.20%) | 1.00 (0.20%) |
| Total | 490.00 | 490.00 |

*approximate molecular weight of 5,000,000

Six (6) of the 5 mg tablets of Example 2 were tested using a USP Apparatus Type 2, at 50 rpms with 500 ml of purified water at 37° C. The average dissolution values are as follows:

| | Oxycodone Hydrochloride |
|---|---|
| Time (minutes) | Tablets 5 mg |
| 0 | 0% |
| 5 | 86% |
| 10 | 91% |
| 15 | 91% |
| 30 | 92% |
| 45 | 93% |

### Example 3

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.00%) | 7.50 (1.49%) |
| Polyethylene oxide | 40.00 (7.96%) | 40.00 (7.96%) |
| (POLYOX WSR 1105*) | | |
| Microcrystalline cellulose | 292.25 (58.19%) | 289.75 (57.69%) |
| (AVICEL PH 102) | | |
| Sodium lauryl sulfate, NF | 7.00 (1.39%) | 7.00 (1.39%) |
| Corn Starch, NF (Uni-Pure ® F) | 100.00 (19.91%) | 100.00 (19.91%) |
| Sodium Bicarbonate, USP | 10.00 (1.99%) | 10.00 (1.99%) |
| Citric Acid, USP | 5.00 (1.00%) | 5.00 (1.00%) |
| Lactose Monohydrate, NF | 27.25 (5.43%) | 27.25 (5.43%) |
| Modified Spray Dried | | |
| (Lactose 316 Fast Flo) | | |
| Colloidal Silicon Dioxide, NF | 2.50 (0.50%) | 2.50 (0.50%) |
| (Cab-O-Sil M5P) | | |
| Magnesium Stearate, NF | 1.00 (0.20%) | 1.00 (0.20%) |
| Subtotal (core) | 490.00 | 490.00 |
| OPADRY II Orange 85F93062 | 12.25 (2.44%) | — |
| OPADRY II Yellow 85F12264 | — | 12.25 (2.44%) |
| Total (coated tablet) | 502.25 (100%) | 502.25 (100%) |

*approximate molecular weight of 900,000

The tablets were prepared by screening the oxycodone hydrochloride, polyethylene oxide and about 1% of the microcrystalline cellulose through a 30 mesh screen, loading the screened material into a V-blender and blending the material for approximately 5 minutes. Approximately one third of the microcrystalline cellulose was screened through a 30 mesh screen and loaded into the V-blender and blended for approximately 10 additional minutes. The remaining microcrystalline cellulose, sodium lauryl sulfate, corn starch, sodium bicarbonate, citric acid, lactose and colloidal silicon dioxide is screened through a 30 mesh screen and loaded into the V-blender and blended for approximately 10 additional minutes. The magnesium stearate was screened through a 30 mesh screen and loaded into the V-blender and blended for approximately 5 additional minutes. The final blend is compressed into tablets with a target hardness of about 5 to 15 kp, preferably with a target hardness of about 12 kp.

The tablets were subsequently coated with an aqueous solution of OPADRY II Orange 85F93062 or OPADRY II Yellow 85F12264.

Twelve coated tablets were tested using a USP Apparatus Type 2, 50 rpms with 500 ml of purified water at 37° C. The average dissolution values are as follows:

| | Oxycodone Hydrochloride | Oxycodone Hydrochloride |
|---|---|---|
| Time (minutes) | Tablets 5 mg | Tablets 7.5 mg |
| 0 | 0% | 0% |
| 5 | 62% | 58% |
| 15 | 75% | 73% |
| 30 | 80% | 78% |
| 45 | 83% | 80% |
| 60 | 84% | 81% |

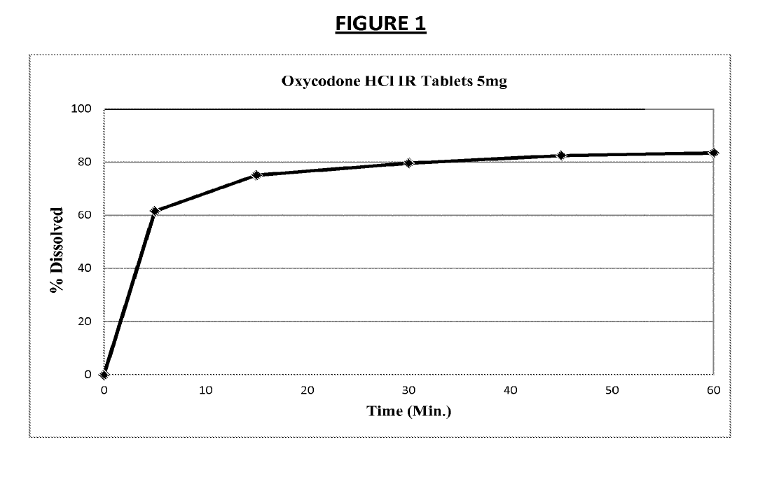

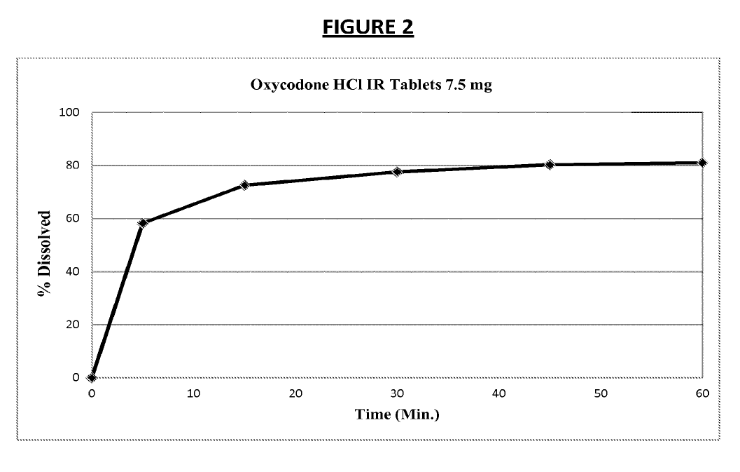

Graphs of the dissolution profile for the 5 mg and 7.5 mg tablets are shown in FIGS. 1 and 2 respectively

### Example 4

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.00%) | 7.50 (1.49%) |
| Polyethylene oxide (POLYOX | 27.50 (5.48%) | 27.5.0 (5.48%) |
| WSR Coagulant, fine powder*) | | |
| Microcrystalline cellulose | 291.50 (58.04%) | 289.00 (57.54%) |
| (AVICEL PH 102) | | |
| Sodium lauryl sulfate, NF | 7.00 (1.39%) | 7.00 (1.39%) |
| Corn Starch, NF | 100.00 (19.91%) | 100.00 (19.91%) |
| (Uni-Pure ® F) | | |
| Sodium Bicarbonate, USP | 18.00 (3.58%) | 18.00 (3.58%) |
| Citric Acid, USP | 9.00 (1.79%) | 9.00 (1.79%) |
| Lactose Monohydrate, NF | 28.50 (5.67%) | 28.50 (5.67%) |
| Modified Spray Dried | | |
| (Lactose 316 Fast Flo) | | |
| Colloidal Silicon Dioxide, NF | 2.50 (0.50%) | 2.50 (0.50%) |
| (Cab-O-Sil M5P) | | |
| Magnesium Stearate, NF | 1.00 (0.20%) | 1.00 (0.20%) |
| Subtotal (core) | 490.00 | 490.00 |
| OPADRY II Orange 85F93062 | 12.25 (2.44%) | — |
| OPADRY II Yellow 85F12264 | — | 12.25 (2.44%) |
| Total (coated tablet) | 502.25 (100%) | 502.25 (100%) |

*approximate molecular weight or 5,000,000

The tablets are prepared according to the procedure outlined in Example 3.

Twelve coated tablets were tested using a USP apparatus Type 2, at 50 rpms with 500 ml of purified water at 37° C. The average dissolution values are as follows:

| | Oxycodone Hydrochloride | Oxycodone Hydrochloride |
|---|---|---|
| Time (minutes) | Tablets 5 mg | Tablets 7.5 mg |
| 0 | 0% | 0% |
| 45 | 83% | 88% |

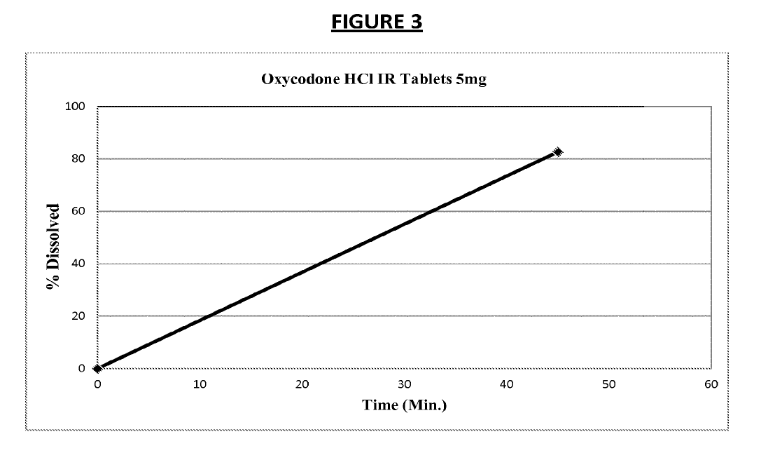

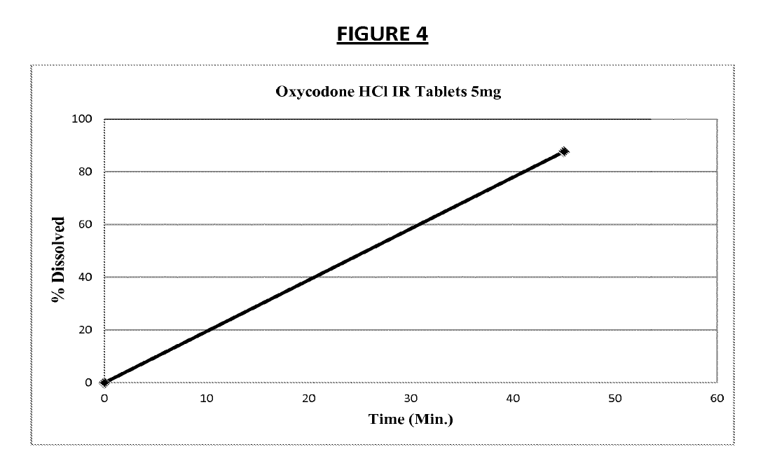

Graphs of the dissolution profile for the 5 mg and 7.5 mg tablets are shown in FIGS. 3 and 4 respectively

### Example 5

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.00%) | 7.50 (1.49%) |
| Polyethylene oxide | 6.25 (1.24%) | 6.25 (1.24%) |
| (POLYOX WSR 1105*) | | |
| Polyethylene oxide (POLYOX | 18.75 (3.73%) | 18.75 (3.73%) |
| WSR Coagulant, fine powder**) | | |
| Microcrystalline cellulose | 297.25 (59.18%) | 294.75 (58.69%) |
| (AVICEL PH 102) | | |
| Sodium lauryl sulfate, NF | 7.00 (1.39%) | 7.00 (1.39%) |
| Corn Starch, NF | 100.00 (19.91%) | 100.00 (19.91%) |
| (Uni-Pure ® F) | | |
| Sodium Bicarbonate, USP | 12.50 (2.49%) | 12.50 (2.49%) |
| Citric Acid, USP | 6.25 (1.24%) | 6.25 (1.24%) |
| Lactose Monohydrate, NF | 33.50 (6.67%) | 33.50 (6.67%) |
| Modified Spray Dried | | |
| (Lactose 316 Fast Flo) | | |
| Colloidal Silicon Dioxide, | 2.50 (0.50%) | 2.50 (0.50%) |
| NF (Cab-O-Sil M5P) | | |
| Magnesium Stearate, NF | 1.00 (0.20%) | 1.00 (0.20%) |
| Subtotal (core) | 490.00 | 490.00 |
| OPADRY II Orange 85F93062 | 12.25 (2.44%) | — |
| OPADRY II Yellow 85F12264 | — | 12.25 (2.44%) |
| Total (coated tablet) | 502.25 (100%) | 502.25 (100%) |

*approximate molecular weight of 900,000
**approximate molecular weight of 5,000,000

The tablets are prepared according to the procedure outlined in Example 3.

Twelve coated tablets were tested using a USP Apparatus Type 2, at 50 rpms with 500 ml of purified water at 37° C. The average dissolution values are as follows:

| | Oxycodone Hydrochloride | Oxycodone Hydrochloride |
|---|---|---|
| Time (minutes) | Tablets 5 mg | Tablets 7.5 mg |
| 0 | 0% | 0% |
| 5 | 68% | 72% |
| 15 | 78% | 81% |
| 30 | 81% | 83% |
| 45 | 84% | 85% |
| 60 | 85% | 86% |

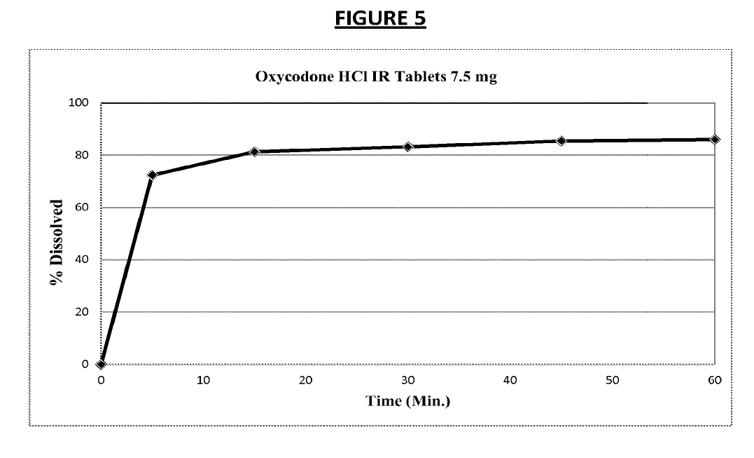

A graph of the dissolution profile for the 7.5 mg tablet is shown in FIG. 5.

### Example 6

Immediate release oxycodone hydrochloride tablets with the following composition were prepared:

| | Mg/Tablet | Mg/Tablet |
|---|---|---|
| Ingredient | (% w/w) | (% w/w) |
| Oxycodone hydrochloride | 5.00 (1.00%) | 7.50 (1.49%) |
| Polyethylene oxide | 5.00 (1.00%) | 5.00 (1.00%) |
| (POLYOX WSR 1105*) | | |
| Polyethylene oxide (POLYOX | 20.00 (3.98%) | 20.00 (3.98%) |
| WSR Coagulant, fine powder**) | | |
| Microcrystalline cellulose | 297.25 (59.18%) | 294.75 (58.69%) |
| (AVICEL PH 102) | | |
| Sodium lauryl sulfate, NF | 7.00 (1.39%) | 7.00 (1.39%) |
| Corn Starch, NF | 100.00 (19.91%) | 100.00 (19.91%) |
| (Uni-Pure ® F) | | |
| Sodium Bicarbonate, USP | 12.50 (2.49%) | 12.50 (2.49%) |
| Citric Acid, USP | 6.25 (1.24%) | 6.25 (1.24%) |
| Lactose Monohydrate, NF | 33.50 (6.67%) | 33.50 (6.67%) |
| Modified Spray Dried | | |
| (Lactose 316 Fast Flo) | | |
| Colloidal Silicon Dioxide, | 2.50 (0.50%) | 2.50 (0.50%) |
| NF (Cab-O-Sil M5P) | | |
| Magnesium Stearate, NF | 1.00 (0.20%) | 1.00 (0.20%) |
| Subtotal (core) | 490.00 | 490.00 |
| OPADRY II Orange 85F93062 | 12.25 (2.44%) | — |
| OPADRY II Yellow 85F12264 | — | 12.25 (2.44%) |
| Total (coated tablets) | 502.25 (100%) | 502.25 (100%) |

*approximate molecular weight of 900,000
**approximate molecular weight of 5,000,000

The tablets are prepared according to the procedure outlined in Example 3.

Twelve coated tablets were tested using a USP Apparatus Type 5, at 50 rpms with 500 ml of purified water at 37° C. The average dissolution values are as follows:

| | Oxycodone Hydrochloride | Oxycodone Hydrochloride |
|---|---|---|
| Time (minutes) | Tablets 5 mg | Tablets 7.5 mg |
| 0 | 0% | 0% |
| 45 | 86% | 85% |

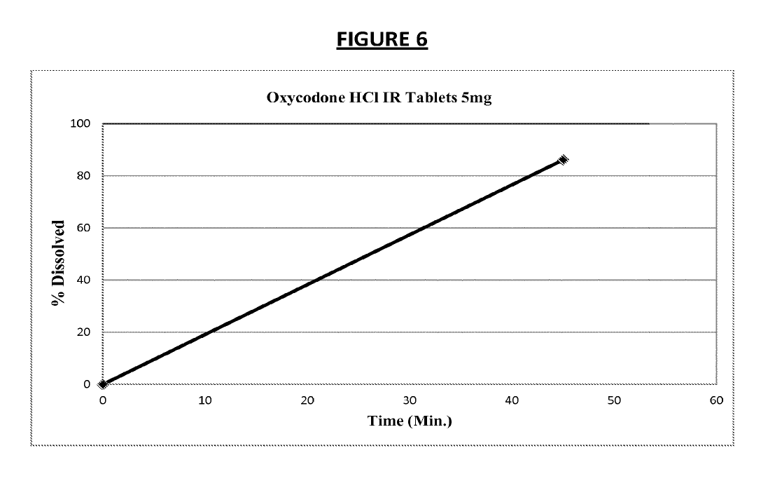

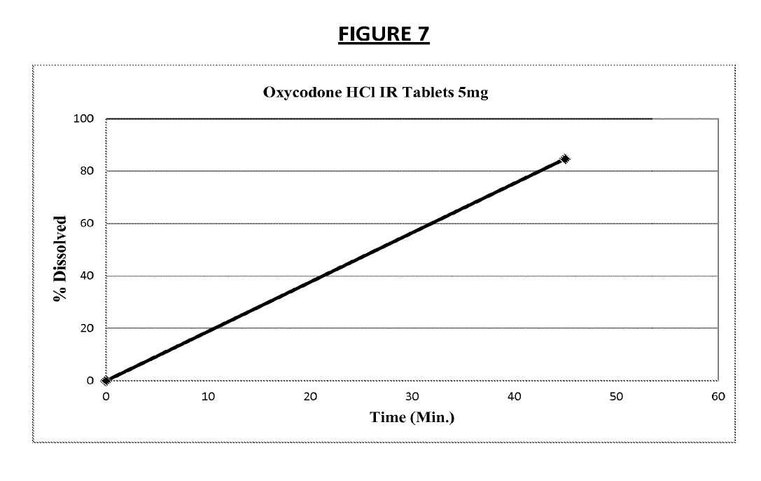

Graphs of the dissolution profile for the 5 mg and 7.5 mg tablets are shown in FIGS. 6 and 7 respectively.

The invention illustratively described herein suitably may be practiced in the absence of any element or elements, limitation or limitations, which is not specifically disclosed herein. Thus, for example, in each instance herein, any of the terms “comprising,” “consisting essentially of” and “consisting of” may be replaced with either of the other two terms. The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1.-23. (canceled)

24. A solid pharmaceutical tablet consisting essentially of:
i) a compressed core; and
ii) optionally an aesthetic or seal coating surrounding the compressed core, wherein the compressed core consists essentially of:
a) a therapeutically effective amount of a drug that is subject to abuse, wherein the drug is selected from the group consisting of alfentanil, alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, zolpidem and pharmaceutically acceptable salts thereof;
b) about 1 to about 20 weight percent of a gelling agent selected from the group consisting of polyhydroalkylcellulose having a molecular weight greater than 50,000, a poly(hydroxyalkylmethacrylate) having a molecular weight of from 5,000 to 5,000,000; a poly(vinylpyrrolidone) having a molecular weight of from 100,000 to 3,000,000; a polysaccharide, a carboxyvinyl polymer, a polymer of acrylic acid cross-linked with a polyallyl ether of sucrose; polyacrylamides; polyethylene oxide polymers having a molecular weight of 100,000 to 7,000,000 and combinations thereof;
c) about 1 to about 20 weight percent of an effervescent agent;
d) optionally at least one conventional pharmaceutical processing excipient; and
e) optionally a second aversive agent selected from the group consisting of a second nasal irritant, an antagonist agent, a bittering agents, a visual modifying agent, an emetic agent and combinations of the forgoing,
wherein the tablet releases 40-90% of the drug in 30 minutes and 70-100% of the drug in 45 minutes when measured by a USP Apparatus Type 2 dissolution test at 50 rpms placed into 500 ml of purified water at 37° C.

25. The tablet as defined in claim 24 wherein at least one conventional pharmaceutical processing excipient is present, and is selected from the group consisting of fillers, binders, lubricants, glidants, disintegrants, coloring agents, and mixtures thereof.

26. The tablet as defined in claim 24 wherein the drug is selected from the group consisting of buprenorphine, codeine, dihydrocodeine, dihydromorphine, hydrocodone, hydromorphone, morphine, oxycodone, oxymorphone and pharmaceutically acceptable salts therefore.

27. The tablet of claim 24 wherein the drug is oxycodone or a pharmaceutically acceptable salt thereof.

28. The tablet as defined in claim 24 wherein the gelling agent is a polyethylene oxide with an approximate molecular weight of about 100,000 to about 7,000,000.

29. The tablet as defined in the claim 28 wherein the gelling agent is a polyethylene oxide with an approximate molecular weight of about 900,000 to about 5,000,000.

30. The tablet as defined in claim 24 wherein the gelling agent is a combination of two or more gelling agents selected from the group consisting of polyethylene oxide, hydroxypropyl cellulose with a molecular weight of about 50,000 to about 125,000, hydroxypropyl methylcellulose with a 2% (w/v) aqueous viscosity at 20° C. between about 50 mPa·s and about 100,000 mPa·s, and polyvinylpyrrolidone with a molecular weight between 400,000 to about 3,000,000.

31. The tablet as defined in claim 24 wherein the combination of two or more gelling agents comprises at least two different types of polyethylene oxides wherein the first polyethylene oxide has an approximate molecular weight between 500,000 and 1,000,000 and the second polyethylene oxide has an approximate molecular weight between 2,000,000 and 5,000,000.

32. The tablet as defined in claim 30 wherein the effervescent agent comprises an alkaline source and an acid source.

33. The tablet as defined in claim 32 wherein the alkaline source is a carbonate or bicarbonate and the acid source is an organic acid or salt of an organic acid.

34. The tablet as defined in claim 24 which exhibits the following release profile when measured by the USP Apparatus Type 2 dissolution test at 50 rpms placed into 500 ml of purified water at 37° C.:
50-80% of the drug is released at 30 minutes; and
80-100% of the drug is released at 45 minutes.

35. The tablet as defined in claim 1 wherein a second aversive agent is present

36. A solid pharmaceutical tablet consisting essentially of:
i) a compressed core; and
ii) optionally an aesthetic or seal coating surrounding the compressed core, wherein the compressed core consists essentially of:
a) a therapeutically effective amount of a drug selected from the group consisting of buprenorphine, codeine, dihydrocodeine, dihydromorphine, hydrocodone, hydromorphone, morphine, oxycodone, oxymorphone and pharmaceutically acceptable salts there of;
b) about 1 to about 20 weight percent of a polyethylene oxide polymer having a molecular weight of 100,000 to 7,000,000;
c) about 1 to about 20 weight percent of an effervescent agent wherein the effervescent agent consists essentially of an alkaline source selected from the group consisting of a carbonate, bicarbonate and a mixture thereof and an acid source selected from the group consisting of an organic acid, a salt of an organic acid and a mixture thereof;
d) at least one conventional pharmaceutical processing excipient selected from the group consisting of fillers, binders, lubricants, glidants, disintegrants, coloring agents, and mixtures thereof;
e) optionally a second aversive agent selected from the group consisting of a second irritating agent, an antagonist agent, a bittering agent, a visual modifying agent, an emetic agent and combinations of the forgoing;
and wherein the tablet releases 40-90% of the drug in 30 minutes and 70-100% of the drug in 45 minutes when measured by a USP Apparatus Type 2 dissolution test at 50 rpms placed into 500 ml of purified water at 37° C.

37. The tablet as defined in claim 36 which exhibits the following release profile when measured by the USP Apparatus Type 2 dissolution test at 50 rpms placed into 500 ml of purified water at 37° C.:
50-80% of the drug is released at 30 minutes; and
80-100% of the drug is released at 45 minutes.

38. The tablet as defined in claim 24 having a hardness from about 5 kp to about 20 kp.

39. The tablet as defined in claim 36 having a hardness from about 5 kp to about 20 kp.
